(19) United States
(12) Patent Application Publication
(10) Pub. No.: US 2014/0309203 A1
(43) Pub. Date: Oct. 16, 2014

(54) PHYTOECDYSONES FOR USE IN WEIGHT STABILIZATION AFTER A WEIGHT-LOSS DIET

(71) Applicants: INSTITUT BIOPHYTIS SAS, ROMAINVILLE (FR); UNIVERSITE PIERRE ET MARIE CURIE, PARIS (FR)

(72) Inventors: Rene Lafont, Paris (FR); Karine Clement, Paris (FR); Salwa Rizkalla, Rungis (FR); Stanislas Veillet, Savigny Sur Orge (FR); Anne-Sophie Foucault, Paris (FR); Waly Dioh, Bretigny Sur Orge (FR)

(21) Appl. No.: 14/356,646

(22) PCT Filed: Nov. 12, 2012
(86) PCT No.: PCT/FR2012/052600

§ 371 Date: May 7, 2014

(30) Foreign Application Priority Data
Nov. 10, 2011 (FR) 1160280

(51) Int. Cl. A61K 31/575; A61K 36/21

(52) U.S. Cl. CPC A61K 31/575; A61K 36/21
USPC 514/181

(57) ABSTRACT

Phytoecdysones to avoid weight gain in obese mammals previously having undergone a hypocaloric weight-loss diet. The phytoecdysones are added to a food composition. The phytoecdysones can be from plants, such as quinoa.

### TECHNICAL FIELD OF THE INVENTION

The invention relates to phytoecdysones, whether in pure form or contained in an extract, for their use in weight stabilization after a weight-loss diet.

More specifically, the invention makes it possible to avoid weight gain in obese mammals having previously undergone a weight-loss diet.

### PRIOR ART

These days, overweight and obesity are physiopathological conditions that are steadily becoming more prevalent throughout the world. In Europe and the USA, an individual is considered overweight when his or her body mass index (BMI) is greater than 25. An individual is considered obese when his or her BMI is greater than 30.

This physiological disorder may be the source of many health complications. For example, metabolic syndrome is a physiological disturbance most often associated with being overweight. A person is recognized as having metabolic syndrome when he or she has at least three of the following five clinical signs associated with said condition:

visceral or abdominal obesity, hypertriglyceridemia, atherogenic dyslipidemia, hypertension, and hyperglycemia (Isomaa et al., 2001). Obesity itself also increases the risk of developing type 2 diabetes (or adult-onset diabetes) and cardiovascular diseases (Rexrode et al., 1998).

One method for an obese mammal to lose weight and body fat is to follow a hypocaloric diet, possibly in combination with drugs and/or dietary supplements, as well as regular physical activity. However, after an initial phase of weight loss, it is common to observe weight regain in individuals on a hypocaloric diet (Ulen et al., 2008). This phenomenon is known as the “rebound effect”.

Many dietary supplements and functional foods have been developed to promote weight loss in overweight mammals (Saper et al., 2004) and to prevent the development of diabetes (McWorther, 2001) and cardiovascular diseases. Most dietary products currently on the market are not effective enough on abdominal obesity, and in some cases, they were found to be toxic (Pittler et al., 2005). Furthermore, they lack the efficacy to prevent the rebound effect after a certain period of time on a hypocaloric diet.

It is therefore necessary to identify new natural molecules, already present in the diet of mammals, in order to develop ingredients and functional foods that are non-toxic and present long-term efficacy on body fat.

Phytoecdysones are plant-derived ecdysteroids. They are natural molecules that belong to the triterpene family and are relatively abundant in the plant kingdom, where they can be found in 5% of wild plants (Báthori and Pongracs, 2005).

As described in patent FR2924346 on behalf of the Applicant, phytoecdysones, and particularly 20-hydroxyecdysone, are known to reduce the increase of body fat in mammals on a fattening hypercaloric diet.

In addition, said molecules have antioxidant properties (Kuzmenko et al., 2001) and lack toxicity (Ogawa et al., 1974).

### DESCRIPTION OF THE INVENTION

The inventors discovered that ingesting phytoecdysones, by obese mammals, can reduce weight regain following an initial phase of weight loss due to a hypocaloric diet.

More specifically, individuals being treated with phytoecdysones stabilize their weight, or even continue losing weight, over the course of a phase known as the stabilization phase, during which a control group regains weight.

Furthermore, during this stabilization phase, individuals being treated with phytoecdysones had a smaller adipocyte size than those in the control group. They also had lower blood insulin levels and greater insulin sensitivity than those in the control group.

The invention therefore proposes to implement phytoecdysones, specifically 20-hydroxyecdysone, to prevent weight regain in obese mammals on a hypocaloric weight-loss diet.

Preferably, phytoecdysones are also used to stabilize, or even continue to reduce, the diameter of adipocytes during the stabilization phase after a hypocaloric weight-loss diet.

Preferably, phytoecdysones are also used to stabilize insulin sensitivity that has improved due to a prior hypocaloric weight-loss diet. In addition to their effect in treating obesity, phytoecdysones are therefore promising in the treatment of diabetes, particularly type 2 diabetes.

The phytoecdysones that are used can be obtained by extraction from plants. Ecdysones prepared by hemisynthesis can also be used.

Phytoecdysones are preferably selected from 20-hydroxyecdysone, makisterone A, 24-epimakisterone A, 24(28)-dehydromakisterone A, 20,26-dihydroxyecdysone, and combinations of two or more of these components.

The phytoecdysones can be in pure form or contained in a more or less enriched plant extract. Advantageously, the phytoecdysones implemented according to the invention are in the form of a plant extract enriched with phytoecdysones, said extract containing at least 1% phytoecdysones by weight. Preferably, the extract contains between 1% and 7% phytoecdysones, more preferably between 1.5% and 3%, and even more preferably 2% phytoecdysones by weight.

Food plants from which the extracts according to the invention come are advantageously selected from chenopodiacae, particularly quinoa and spinach (Findeisen, 2004). Medicinal plants can also be used to develop extracts that are rich in phytoecdysones.

Preferably, a plant extract enriched with phytoecdysones according to the invention comes from quinoa. Quinoa is an edible pseudo-cereal that is rich in phytoecdysones (Zhu et al., 2001; Dini et al., 2005). It is thus possible to supplement food by ingesting extract of quinoa that has been enriched with phytoecdysones, by introducing said extract into a food, such as a milk product or a beverage, or by consuming it as a dietary supplement, such as in the form of a capsule.

Quinoa is currently known as the food plant that is the richest in phytoecdysones. Quinoa seeds contain a blend of phytoecdysones (Zhu et al., 2001). Said phytoecdysones are especially abundant in the shell of the quinoa seeds. For example, a 60-gram lot of quinoa seeds (dry weight) provides 15 to 25 milligrams of 20-hydroxyecdysone.

Phytoecdysones implemented according to the invention are advantageously in the form of a composition that can be orally administered.

Composition means, for example, a food product, such as a beverage, a milk product, or something else. Of course, the composition can be a medicinal composition, such as one used in the form of pills which thus contain an exact dose of phytoecdysones.

Advantageously, the phytoecdysones are orally administered at a rate of 0.3 to 2.0 mg per kg of body weight per day, preferably 0.5 mg per kg per day.

Another purpose of the invention is a method for preparing a quinoa extract enriched with one or more phytoecdysones, said method comprising the following steps:
water extraction of quinoa seeds;
solid/liquid separation and centrifugation of the aqueous extract;
heating of the supernatant so as to precipitate proteins;
purification by chromatography of the supernatant so as to enrich it with phytoecdysones.

The invention also relates to the quinoa extract resulting from the aforementioned method. Said extract can advantageously be used to avoid weight regain in obese mammals on a hypocaloric weight-loss diet, as described above.

### BRIEF DESCRIPTION OF THE DRAWINGS

FIGS. 1A, 1B: Graphs representing the weight change in obese individuals on a hypocaloric diet;

FIGS. 2A, 2B: Graphs representing the change in adipocyte diameter in obese individuals on a hypocaloric diet;

FIGS. 3A, 3B: Graphs representing the change in insulin levels in obese individuals on a hypocaloric diet;

FIGS. 4A, 4B: Graphs representing the change in insulin sensitivity in obese individuals on a hypocaloric diet;

FIG. 5: The chemical formulas of phytoecdysones present in a composition according to an embodiment of the invention.

### DETAILED DESCRIPTION

In the invention, it is proposed to provide a dose of phytoecdysones in the form of purified molecules, or by means of a plant extract enriched with phytoecdysones, so as to prevent weight regain, known as the “rebound effect”, in obese mammals on a hypocaloric weight-loss diet.

According to the invention, it is possible to provide said dose of phytoecdysones in the form of a plant extract, such as quinoa, incorporated for example in food added to an individual's daily diet. One gram of quinoa that has been enriched with up to 2% phytoecdysones by weight contains 20 milligrams of phytoecdysones. To obtain the same quantity of phytoecdysones from quinoa seeds, it would be necessary to consume 50 grams of unprocessed quinoa seeds (Dini et al., 2005; Kumpun et al., 2011). The quinoa extract according to the invention may also contain up to 50 times more phytoecdysones than the quinoa seeds from which it is produced.

I—An example of a method for preparing the quinoa extract enriched with phytoecdysones (Extract A)

The quinoa seeds are first ground to separate the flour from the bran of the seed. An extraction is then carried out by adding 4,000 L to 400 kg of bran. The aqueous extract undergoes solid/liquid separation, followed by centrifugation. The resulting supernatant is subjected to 90° C. heat to precipitate the proteins. The aqueous extract is then purified by being passed through a column of food resin for the purpose of enriching it with phytoecdysones. The ethanol eluate is then dried by means of spray drying after adding a bit of maltodextrin, appropriated to adjust the 2±0.2% content in weight of 20-hydroyecdysone (20E).

Such a sequential extraction makes it possible to eliminate saponins from the extract, which are abundant in quinoa seeds (Muir et al., 2002) and which would create a bitter taste in said extract, and most sugars.

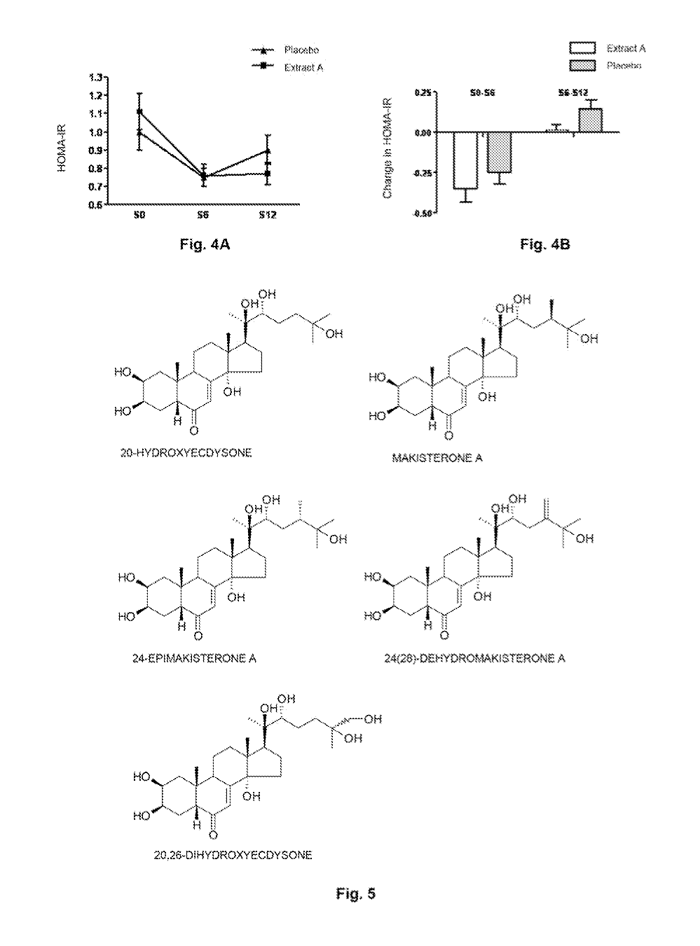

The obtained extract contains a blend of phytoecdysones, of which 85-90% is 20-hydroxyecdysone. The remainder is comprised of other very similar phytoecdysones, such as makisterone A, 24-epimakisterone A, 24(28)-dehydromakisterone A, 20,26-dicydroxyecdysone (Kumpun et al, 2011). The structures of these components are shown in FIG. 5.

An extract similar to extract A, usable as part of the invention, is notably sold under the number Quinolia®.

II—A double-blind clinical trial on the effects of extract A on obese individuals on a hypocaloric diet for six weeks followed by a stabilization diet for six weeks

### Protocol

The effect of extract A was studied as part of a double-blind clinical trial on 60 overweight and obese volunteers, comprised of 18 men and 42 women with a BMI ranging from 27 to 38. The volunteers were given a hypocaloric diet of 1200 kcal for women and 1500 kcal for men for six weeks. The hypocaloric diet is followed by a six-week stabilization diet, with 20% more calories than the hypocaloric diet. Parameters such as insulin levels, weight change, adipocyte diameter, and muscle strength were measured during the visits at the start and end of the hypocaloric and stabilization phases.

Subjects were split into two groups. A first group (“extract A” group) received six capsules of extract A, containing a total of 40 mg of phytoecdysones, in three daily doses, throughout the duration of the test.

A second control group (“placebo” group) received six capsules of placebo in three daily doses, for the same duration.

Measurements were taken at the beginning of treatment (W0), at the end of the sixth week of the diet (W6), and at the end of the twelfth week of the diet (W12).

### Measurement of Weight Change During the Weight-loss and Stabilization Diets

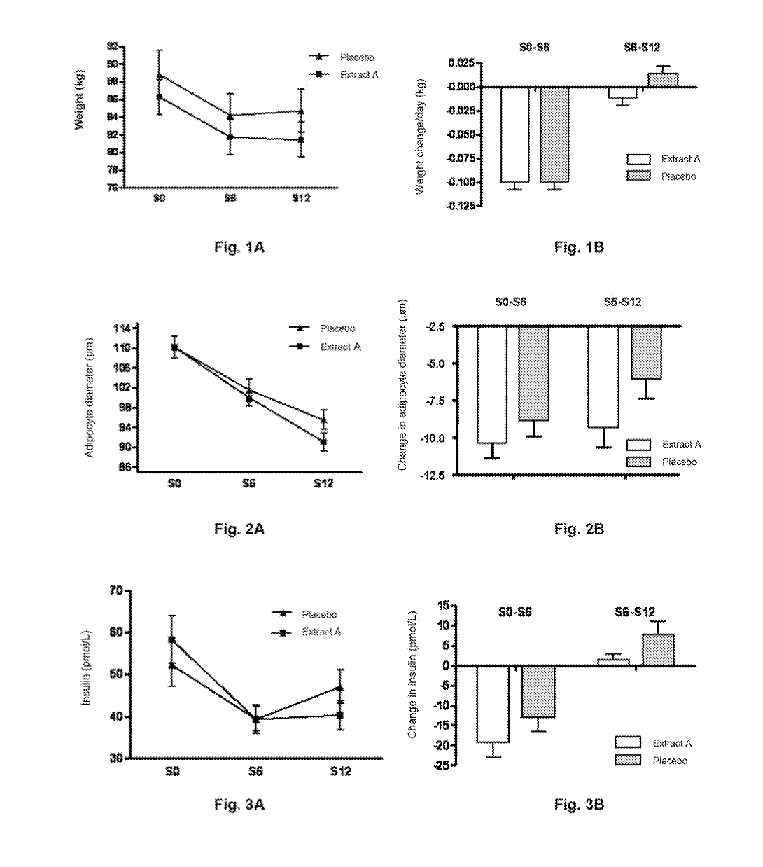

The effect of extract A during a hypocaloric diet was studied based on weight change (FIGS. 1A and 1B).

The “placebo” and “extract A” groups showed respective weight losses of 4.05 kg and 3.86 kg during the first phase of the diet, called the weight-loss phase.

During the second phase of the diet, called the stabilization phase, the “extract A” group continued to lose weight (−0.483 kg), while the “placebo” group posted an average gain of +0.504 kg.

The study of relative weight change in the tested individuals shows that, in the “extract A” group, only 10% of individuals rebounded (a gain of 0.05 to 0.1 kg/day), compared to 28% of the “placebo” group.

The same “extract A” group had 20% of individuals who had lost 0.06 to 0.15 kg/day, while in the “placebo” group, only 10% of individuals fell into that category.

### Measurement of Adipocyte Diameter During the Weight-loss and Stabilization Diets

The effect of extract A during a hypocaloric diet was studied based on adipocyte diameter change (FIGS. 2A and 2B).

During the weight-loss phase, the average adipocyte diameter was reduced both in the “extract A” group (−10.94 μm) and in the “placebo” group (−8.80 μm).

During the stabilization phase, the average diameter continued to decline, with a significant difference between the “extract A” group (−9.25 μm) and the “placebo” group (−5.99 μm) group.

This result is combined with a greater loss in body fat during the stabilization phase in the “extract A” group. In fact, the loss of body fat during the stabilization phase was −0.74 kg for the “extract A” group, but only −0.33 kg for the “placebo” group.

### Measurement of Insulin Levels and Insulin Resistance During the Weight-loss and Stabilization Diets

The effect of extract A during a hypocaloric diet was studied based on the change in insulin levels (FIGS. 3A and 3B) and insulin resistance (FIGS. 4A and 4B). The HOMA-IR (Homeostasis Model Assessment of Insulin Resistance, FIGS. 4A and 4B) index is a marker of insulin resistance.

Blood insulin levels declined during the weight-loss phase in the “extract A” group (−18.90 pmol/L) and the “placebo” group (−12.60 pmol/L). They climbed back up during the stabilization phase in the “placebo” group (7.69 pmol/L), but they stabilized in the “extract A” group. The treatment with extract A makes it possible to stability blood insulin levels in the stabilization phase and to decrease them significantly more than placebo throughout treatment.

Insulin resistance, measured with the HOMA-IR index, declined in both groups during the weight-loss phase. It remained constant in the “extract A” group during the stabilization phase, but it went back up significantly in the “placebo” group.

### Conclusions

The administration of extract A makes it possible for object subjects to avoid weight regain during the second phase, called the stabilization phase, of the hypocaloric weight-loss diet.

Furthermore, the administration of extract A allows for a more significant decline in body fat and average adipocyte diameter during the stabilization phase.

The administration of extract A finally makes it possible to lower insulin levels and maintain the improved insulin sensitivity brought about by the hypocaloric diet during this same phase.

### REFERENCES

Báthori M, Pongrácz Z. 2005. Phytoecdysteroids—from isolation to their effects on humans. Current Medicinal Chemistry 12, 153-172.
Dini I, Tenore G C, Dini A. 2005. Nutritional and antinutritional composition of Kancolla seeds: An interesting and underexploited andine food plant. Food Chemistry, 92, 125-132.
Findeisen E. 2004. Ecdysteroids in Human Food (Ecdysteroide in der menschlichenNahrung).PhD thesis, University of Marburg, Germany.
Isomaa B, Almgren P, Tuomi T, Forsen B, Lahtii K, Nissen M, Taskinen M R, Groop L. 2001. Cardiovascular morbidity and mortality associated with the metabolic syndrome. Diabetes Care 24(4), 683-689.
Kumpun S, Maria A, Crouzet C, Evrard-Todeschi N, Girault J P, Lafont R. 2011. Ecdysteroids from Chenopodium quinoaWilld., an ancient Andean crop of high nutritional value, Food Chemistry 125, 1226-1234.
Kuzmenko A I, Niki E, Noguchi N. 2001. New functions of 20-hydroxyecdysone in lipid peroxidation. Journal of Oleo Science 50, 497-506.
McWorther L S. 2001. Biological complementary therapies: a focus on botanical products in diabetes. Diabetes Spectrum 14, 199-208.
Ogawa S, Nishimoto N, Matsuda H. 1974. Pharmacology of ecdysones in vertebrates.

In: Invertebrate Endocrinology and Hormonal Heterophylly (Burdette, W. J. ed.) Springer, New York.341-344.
Pittler M H, Schmidt K, Ernst E. 2005. Adverse events of herbal food supplements for body weight reduction: systematic review. Obesity Review 6, 93-111.
Rexrode K, Carey V, Hennekens C H, Walters E E, Colditz G A, Stampfer M J, Willet W C,
Manson J E. 1998. Abdominal adiposity and coronary heart disease in women. JAMA 280, 1843-1848.
Saper R B, Eisenberg D M, Phillips R S. 2004. Common dietary supplements for weight loss. American Family Physician 70(9), 1731-1738.
Ulen C G, Huizinga M, Beech B, Elasy T A. 2008. Weight regain prevention. Clinical Diabetes 26(3), 100-113.
Zhu N, Kikusaki H, Vastano B C, Nakatani N, Karwe M V, Rosen R T, Ho C T. 2001. Ecdysteroids of quinoa seeds (Chenopodium quinoaWilld.). Journal of Agricultural and Food Chemistry 49, 2576-2578.

1. Phytoecdysones for their use in order to prevent weight regain in mammals after a hypocaloric weight-loss diet.

2. Phytoecdysones according to claim 1, for their use in order to stabilize the diameter of adipocytes after a hypocaloric weight-loss diet.

3. Phytoecdysones according to claim 1, for their use in order to stabilize the insulin sensitivity previously improved by a hypocaloric weight-loss diet.

4. Phytoecdysones according to claim 1, such that they are selected from 20-hydroxyecdysone, makisterone A, 24-epimakisterone A, 24(28)-dehydromakisterone A, 20,26-dihydroxyecdysone, and combinations of two or more of these components.

5. Phytoecdysones according to claim 1, provided in the form of a plant extract.

6. Phytoecdysones according to claim 5, such that the extract comprises at least 1% phytoecdysones by weight.

7. Phytoecdysones according to claim 5, such that the plant extract comes from quinoa.

8. Phytoecdysones according to claim 1, incorporated into a composition that can be orally administered.

9. A method for preparing a quinoa extract enriched with one or more phytoecdysones, said method comprising the following steps:
water extraction of quinoa seeds to provide an aqueous extract;
solid/liquid separation and centrifugation of the aqueous extract;
heating of supernatant to precipitate proteins; and
purification by chromatography of the supernatant to enrich it with phytoecdysones.
